(19) United States
(12) Patent Application Publication
(10) Pub. No.: US 2023/0320599 A1
(43) Pub. Date: Oct. 12, 2023

(54) BLOOD PRESSURE DETERMINATION OVER A PERIOD OF TIME

(71) Applicant: BIOINTELLISENSE, INC., Golden, CO (US)

(72) Inventors: David Jonq WANG, Palo Alto, CA (US); James R. MAULT, Evergreen, CO (US); Zongde QIU, Cupertino, CA (US); Henry LEUNG, Golden, CO (US); Grant WELLER, Golden, CO (US); Alexander KATSIS, Golden, CO (US)

(73) Assignee: BIOINTELLISENSE, INC., Golden, CO (US)

(21) Appl. No.: 18/299,501

(22) Filed: Apr. 12, 2023

Related U.S. Application Data
(60) Provisional application No. 63/362,878, filed on Apr. 12, 2022.

(51) Int. Cl. A61B 5/021; A61B 5/11; A61B 5/024; A61B 5/00; A61B 5/0245

(52) U.S. Cl. CPC A61B 5/02125; A61B 5/1116; A61B 5/02416; A61B 5/7278; A61B 5/0245

(57) ABSTRACT

The present disclosure generally relates to the determination of blood pressure of a human being. More specifically, but not exclusively, in one aspect the present disclosure relates to determination of blood pressure of a human being over an extended period of time. In one form, a method for determining a blood pressure value of a human being includes determining a period of time during which the human being is stationary. The method also includes determining, during the period of time, a plurality of measurements providing pulse-related data values from the human being. From the plurality of measurements, a representative pulse-related data value representative of the pulse-related data values determined during the period of time may be determined. The method also includes determining, from the representative pulse-related data value, a blood pressure value of the human being for the period of time.

### CROSS REFERENCE TO RELATED APPLICATIONS

The subject application claims priority to U.S. Provisional Patent Application No. 63/362,878 filed Apr. 12, 2022. The contents of this application are incorporated herein by reference in their entirety.

### FIELD

The present disclosure generally relates to the determination of blood pressure of a human being. More specifically, but not exclusively, in one aspect the present disclosure relates to the automatic and periodic measurement of blood pressure of a human being over an extended period of time.

### BACKGROUND

Unless otherwise indicated herein, the materials described herein are not prior art to the claims in the present application and are not admitted to be prior art by inclusion in this section.

Blood pressure values may be determined over a course of time using various pulse-related information and data. For example, an initial calibration value may be established by taking one or more blood pressure readings of a human being through a conventional approach, such as through the use of a blood pressure cuff. The initial calibration value may then be correlated with pulse-related information and data in order to ascertain the pulse-related information and data associated with the initial calibration value. Moving forward, based on this initial calculation, additional pulse-related information and data measurements may be taken and then used to determine a blood pressure value of the human being without measurement through a conventional approach, such as through the use of the blood pressure cuff.

With further reference to FIGS. 1A and 1B for example, and by way of background, pulse-related information and data may be analyzed and then used to generate a blood pressure value by looking at the second derivative of how the blood flow pattern behaves after the arterial pulse. FIG. 1A is a graph of a time-series plot of blood flow observed with a photoplethysmogram (PPG). Taking the second derivative of the blood flow pattern behavior after the arterial pulse, the graph provided in FIG. 1B illustrates a signal which may then be used for determining blood pressure based on the blood flow pattern.

The approach illustrated and described in connection with FIGS. 1A and 1B may be reliable if the measurements taken in connection with the blood flow patterns provide data relating to the blood flow patterns which is of sufficient quality. However, in some circumstances, sensors may provide data relating to the blood flow pattern which lacks in the requisite quality for reliably and consistently determining blood pressure. For example, sensors positioned on certain areas of the human being may provide measurements of less-than-optimal quality. Without limitation for example, the measurements may provide signals which are poor or noisy, and which may not provide accurate blood pressure value determination.

In view of the foregoing, there is a need for additional contributions in this area of technology.

The subject matter claimed herein is not limited to implementations that solve any disadvantages or that operate only in environments such as those described above. Rather, this background is only provided to illustrate one example technology area where some implementations described herein may be practiced.

### SUMMARY

This Summary is provided to introduce a selection of concepts in a simplified form that are further described below in the Detailed Description. This Summary is not intended to identify key features or essential characteristics of the claimed subject matter, nor is it intended to be used as an aid in determining the scope of the claimed subject matter.

In one embodiment, a method for determining a blood pressure value of a human being includes determining a period of time during which the human being is stationary; determining, during the period of time, a plurality of measurements providing pulse-related data values from the human being; determining, from the plurality of measurements, a representative pulse-related data value representative of the pulse-related data values determined during the period of time; and determining, from the representative pulse-related data value, a blood pressure value for the human being during the period of time.

In another embodiment, a system for determining a blood pressure value of a human being includes a first sensor configured to determine a period of time during which the human being is stationary. The system also includes a at least one pulse-related sensor configured to determine a plurality of measurements providing pulse-related data values of the human being during the period of time. A processor is configured to determine, from the plurality of measurements, a representative pulse-related data value representative of the plurality of pulse-related data values determined during the period of time, and to determine, from the representative pulse-related data value, a blood pressure value for the human being during the period of time.

Additional features and advantages of the invention will be set forth in the description which follows, and in part will be obvious from the description, or may be learned by the practice of the invention. The features and advantages of the invention may be realized and obtained by means of the instruments and combinations particularly pointed out in the appended claims. These and other features of the present invention will become more fully apparent from the following description and appended claims, or may be learned by the practice of the invention as set forth hereinafter.

### BRIEF DESCRIPTION OF THE DRAWINGS

To further clarify the above and other advantages and features of the present invention, a more particular description of the invention will be rendered by reference to specific embodiments thereof which are illustrated in the appended drawings. It is appreciated that these drawings depict one non-limiting typical embodiment of the invention and are therefore not to be considered limiting of its scope. The invention will be described and explained with additional specificity and detail through the use of the accompanying drawings in which:

FIGS. 1A and 1B are graphical illustrations of an approach for determining a blood pressure value from pulse-related data and information;

FIG. 2 is a schematic illustration of a system for determining a blood pressure value;

FIG. 3 is a graphical illustration of the correlation of Pulse Transmit Time features of ECG and PPG;

FIG. 4 is a graphical illustration of the correlation of Pulse Arrival Time features of an accelerometer/acoustic sensor and PPG;

FIG. 5 is a graphical illustration of the extraction of morphological features correlated with blood pressure values from a PPG wave;

FIGS. 6 and 7 are graphical illustrations of the integration of multi-channel and/or multi-frame PPG sensor data values;

FIG. 8 is a flowchart of an example method for determining a blood pressure value; and

FIG. 9 illustrates an example computing system.

### DETAILED DESCRIPTION

Reference will now be made to the drawings to describe various aspects of example embodiments of the invention. It is to be understood that the drawings are diagrammatic and schematic representations of such example embodiments, and are not limiting of the present invention, nor are they necessarily drawn to scale.

The present disclosure generally relates to the determination of blood pressure of a human being. More specifically, but not exclusively, in one aspect the present disclosure relates to the automatic and periodic measurement of blood pressure of a human being over an extended period of time. Although various embodiments may be described in the context of use in determining blood pressure values, embodiments disclosed herein may be employed in other fields or operating environments where the functionality disclosed herein may be useful. Accordingly, the scope of the invention should not be construed to be limited to the example implementations and operating environments disclosed herein.

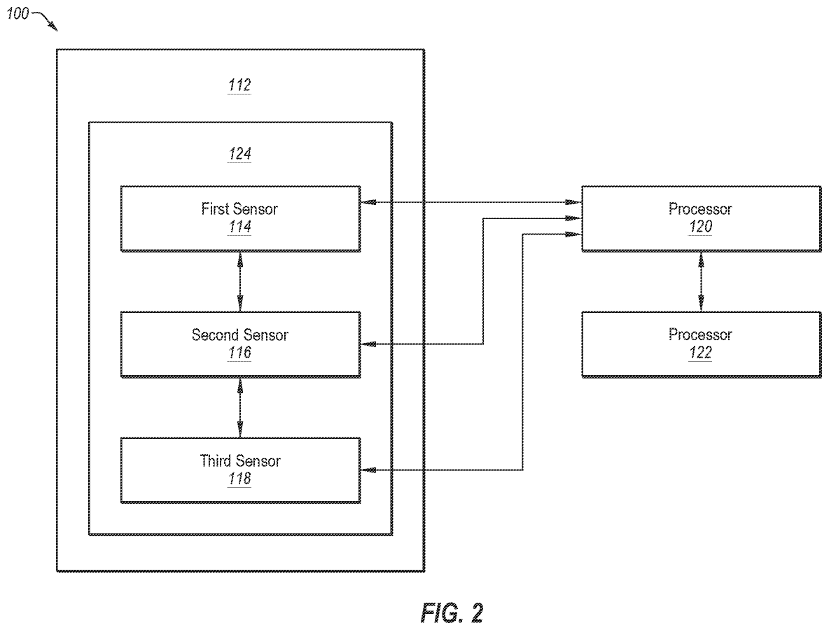

With reference now to FIG. 2, there is schematically illustrated one non-limiting form of a system 100 configured to monitor or determine blood pressure of a human being or human patient 112, although use of the system 100 in connection with non-human subjects is also possible and contemplated. In one form for example, the system 100 may be configured to remotely monitor and determine (e.g., without the need for intervention by a health care professional) blood pressure of a human being or human patient 112 over an extended period of time, or provide a number of blood pressure determinations of the human being or human patient 112 a number of times during the extended period of time. By way of example, the system 100 may be configured to monitor and determine the blood pressure of a human over the course of several days, about a week, or more than a week, although other variations are possible.

In the illustrated form, the system 100 includes a first sensor 114, a second sensor 116, a third sensor 118, a first processor 120, and a second processor 122. The first sensor 114, the second sensor 116 and the third sensor 118 are in communication with the first processor 120 which, in turn, is in communication with the second processor 122. However, variations in the communication arrangement between components of the system 100 are possible and contemplated. For example, one or more of the sensors 114, 116 and 118 may additionally or alternatively communicate with the second processor 122. In one aspect, the first sensor 114 may be an electrocardiogram (ECG) sensor, the second sensor 116 may be a photoplethysmography (PPG) sensor, and the third sensor 118 may be a combined accelerometer and acoustic sensor, although variations in the forms of the sensors 114, 116 and 118 are possible. Forms in which the accelerometer and the acoustic sensor are provided as stand-alone sensing components are also possible, as well as forms in which the system 100 includes sensors in addition to those currently identified. In addition, it should be appreciated that any number of the sensors of the system 100 may include more than one sensor. For example, and without limitation, when the second sensor 116 is a PPG sensor, there may be more than one spectral sensor or photo diode detecting the PPG.

In one form, the first sensor 114, the second sensor 116 and the third sensor 118 may be provided in a common housing 124 configured to be positioned at a location on the human being or human patient 112. In one non-limiting form for example, the housing 124 may be configured for placement on or attachment to the chest of the human being or human patient 112. Forms in which the first sensor 114, the second sensor 116 and the third sensor 118 are not provided in a common housing are also possible, including forms where one or more of the first sensor 114, the second sensor 116 and the third sensor 118 is/are configured to be positioned at different locations on the human being or human patient 112. In the illustrated form, the first sensor 114 and the third sensor 118 are also configured to communicate with the second sensor 116, although forms in which these sensors do not directly communicate with one another, as well as forms in which each of the sensors 114, 116 and 118 is configured to communicate with each of the other sensors, are also possible. In one aspect, operation of one or more of the sensors 114, 116 and 118 may at least be partially dependent on measurements or observations recorded by a different sensor, although forms in which each of the sensors 114, 116 and 118 operates independent of the others are possible.

As indicated above, the system 100 includes a first processor 120 and a second processor 122, although forms in which the system 100 only includes one processor are also contemplated. Generally speaking, the first processor 120 may be configured as an edge computing engine which may, for example, be included in the housing 124, although variations in the location or positioning of the first processor 120 are possible. For example, the first processor 120 may be cloud-based or located on a server, smartphone, tablet or PC, just to provide a few possibilities. The first processor 120 may be configured to determine a blood pressure value for the human patient 112, or an estimated blood pressure value for the human patient 112, based on information provided by one or more of the sensors 114, 116, and 118, further details of which will be provided below. Generally speaking, the second processor 122 may be configured as a cloud-based processor, although variations in the location or positioning of the second processor 122 are possible. For example, the second processor 122 may be hosted on a server or provided on a smartphone, tablet or PC, just to provide a few examples. It is also contemplated that the second processor 122 could be located in the housing 124. The second processor 122 may be configured to determine a blood pressure value for the human patient 112 based on the estimated blood pressure value for the human patient 112 provided by the first processor 120 and/or based on information provided by one or more of the sensors 114, 116, and 118.

In one non-limiting form, the third sensor 118 may be configured to determine motion of the human patient 112, or the system 100 may include an additional, different sensor configured to determine or detect motion of the human patient 112. For example, the third sensor 118 or an additional sensor of the system 100 may include or be provided as an accelerometer, although other variations in the form of the third or additional sensor are contemplated, provided that it may be configured to determine or detect motion, or lack of motion, of the human patient 112. While not previously mentioned, it is contemplated that the third sensor 118 or an additional sensor of the system 100 sensor could be configured to ignore detected movement or motion of the human patient 112 if it does not meet or exceed a predetermined threshold. Stated alternatively, the third sensor 118 or an additional sensor of the system 100 sensor may be configured such that it does not positively identify movement or motion of the human patient 112 unless the movement or motion meet or exceed a predetermined threshold. Additionally or alternatively, the third sensor 118 or an additional sensor of the system 100 sensor may be configured to ignore one or more detected movements or motions that are not observed in combination with other detected movements or motion of the human patient 112. In this manner, the third sensor 118 or an additional sensor of the system 100 sensor may be configured to determine periods of time where the human patient 112 is generally, but not entirely, inactive.

In one aspect, the sensors 114, 116 and 118 may operate at the same time, or operation thereof may alternate. As such, in one form, the first sensor 114, the second sensor 116 and the acoustic sensor of the third sensor 118 may operate regardless of the active/inactive status of the human patient 112. In this form for example, the third sensor 118 or an additional sensor of the system 100 sensor may be configured to identify a period of time in which the human patient 112 is not active (e.g., stationary) by looking back at readings taken thereby to assess when the last recorded movement occurred. The sensor 118 or an additional sensor of the system 100 may be configured to identify any such periods of time only if they meet or exceed a predetermined threshold. For example, the sensor may be configured to indicate a period of inactivity has occurred if five minutes, ten minutes, fifteen minutes, twenty minutes, etc. has passed since the last recorded movement. It should be appreciated that variations in the threshold period of time since the last recorded motion or movement are also possible.

In addition, after the third sensor 118 or an additional sensor of the system 100 sensor determines a threshold period of time has occurred, it may continue monitoring motion or movement of the human patient 112 and when further motion or movement of the human patient is observed, it may consider this further motion or movement as the end the period of time of inactivity. After the motion or movement of the human patient 112 is observed which is considered to end the period of inactivity, the entire period between this observation and the last recorded movement may be considered as the period of time of inactivity. In one example, the period of inactivity determined by the third sensor 118 or an additional sensor of the system 100 may correspond to a time period between a time when the human patient 112 falls asleep and a time when the human patient 112 awakens. As another example, the period of inactivity determined by the third sensor 118 or an additional sensor of the system 100 may correspond to a period of time between a time when the human patient 112 sits down while watching television to the time when the human patient 112 stands up from watching television. It should be appreciated that other variations are possible.

After the third sensor 118 or an additional sensor of the system 100 has determined a period of inactivity has occurred, observations recorded by the first sensor 114, the second sensor 116 and the third sensor 118 during this period of time may be associated with the period of time of inactivity. As such, data provided by one or more of the sensors during this period of time may be used, as will be described in greater detail below, to determine a blood pressure value for the human patient 112 during the period of time of inactivity.

In one form for example, the third sensor 118 or an additional sensor of the system 100 may provide the observed period of time of inactivity to one or both of the first processor 120 and the second processor 122, one or more of the sensors 114, 116 and 118 may provide its recorded observations to one or more of the first processor 120 and the second processor 122 independent of any observed period of time of inactivity, and the first processor 120 and/or the second processor 122 may be configured to identify the recorded observations of the sensors 114, 116 and 118 which occurred during the observed period of time of inactivity. Alternatively, the third sensor 118 or an additional sensor of the system 100 may communicate the observed period of time of inactivity directly to one or more of the sensors 114, 116 and 118, and one or more of the sensors 114, 116 and 118 may then transmit to the first processor 120 and/or the second processor 122 the observations it recorded during the observed period of inactivity. In this latter instance for example, the transmittal of a select portion of the observations recorded by the sensors 114, 116 and 118 may reduce power consumption because the overall amount of information transmitted by the sensors 114, 116 and 118 may be reduced.

In another aspect, the third sensor 118 or an additional sensor of the system 100 may be configured to detect or determine motion, or a lack of motion, of the human patient 112 for a predetermined period of time and then provide an indication or signal to one or more of the sensors 114, 116 and 118 that the predetermined period of time has been satisfied. For example, the third sensor 118 or an additional sensor of the system 100 may provide an indication to one or more of the sensors 114, 116 and 118 that the predetermined period has been satisfied after motion, or a limited amount or degree of motion, of the human patient has not been detected for a number of minutes or hours, although variations are possible and contemplated.

In one aspect, one or more of the sensors 114, 116 and 118 may be configured to forego operation, or operate in less than a full capacity, unless and until a signal from the third sensor 118 or an additional sensor of the system 100 is provided and indicates that the predetermined period of time of inactivity has been satisfied, signaling that the human patient 112 has entered into a period of time of inactivity. In this aspect, one or more of the sensors 114, 116 and 118 may operate, or operate at a full capacity, until motion, or partial motion, of the human patient 112 is again determined, e.g., until the end of the period of time of inactivity. Similarly, the sensing conducted by one or more of the sensors 114, 116, and 118, as well as the related data provided thereby, may be associated with a period of time between when the predetermined period of time of inactivity has been satisfied and until the end of the period of time of inactivity. In this form, one or more of the sensors 114, 116 and 118 may be configured to transmit to the first processor 120 and/or the second processor 122 the data collected during this period of time.

The third sensor 118 or an additional sensor of the system 100 may also be configured to detect or determine the positioning of human patient 112 during the period of inactivity. For example, it may determine if the human patient is sitting up, laying prone, laying supine, or laying on their side, each of which may have a different impact on the blood pressure of the human patient 112. Depending on the positioning of the human patient 112 which is determined, different calibrations or blood pressure determination factors may be implemented by the first processor 120 and/or the second processor 122 in order to account for the different positions of the human patient 112 during the period of inactivity or otherwise.

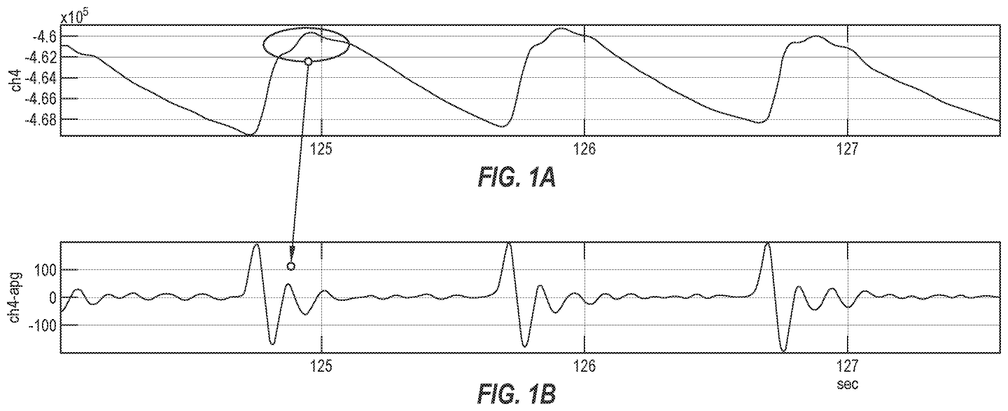

While not previously discussed, it should be appreciated that the first sensor 114, the second sensor 116 and the third sensor 118 may be configured to determine a plurality of measurements providing pulse-related data values of the human patient 112. In one form for example, the first sensor 114 may be an electrocardiogram (ECG) sensor and the second sensor may be a photoplethysmogram (PPG) sensor. In this or other forms, the pulse-related data values provided by the first sensor 114 and the second sensor 116 may be in the form of blood flow patterns or pulse waveforms associated with the arterial pulse of the human patient, although additional or alternative pulse-related data values may be provided by the first sensor 114 and the second sensor 116. Similarly, in these forms, the first processor 120 and/or the second processor 122 may be configured to determine blood pressure of the human patient 112 in a manner similar to that described above in connection with FIGS. 1A and 1B. In some forms, the third sensor 118 may include an accelerometer and an acoustic sensor which are configured to detect and record cardiac mechanical signals.

In one aspect, the system 100 may be generally configured to utilize a number of different pulse-related data information components and values in order to estimate and/or determine the blood pressure of the human patient 112. For example, the system 100 may estimate Pulse Transmit Time (PTT) or Pulse Arrival Time (PAT) features from the ECG sensor of the first sensor 114, the PPG sensor of the second sensor 116, and the accelerometer and the acoustic sensor of the third sensor 118. Additionally or alternatively, the system 100 may estimate Blood Vessel Elastics (BVE) features from PPG signal morphology obtained with the second sensor 116. The system 100 may also be configured to enhance feature quality utilizing multi-channel and multi-beat integration as will be described in greater detail below.

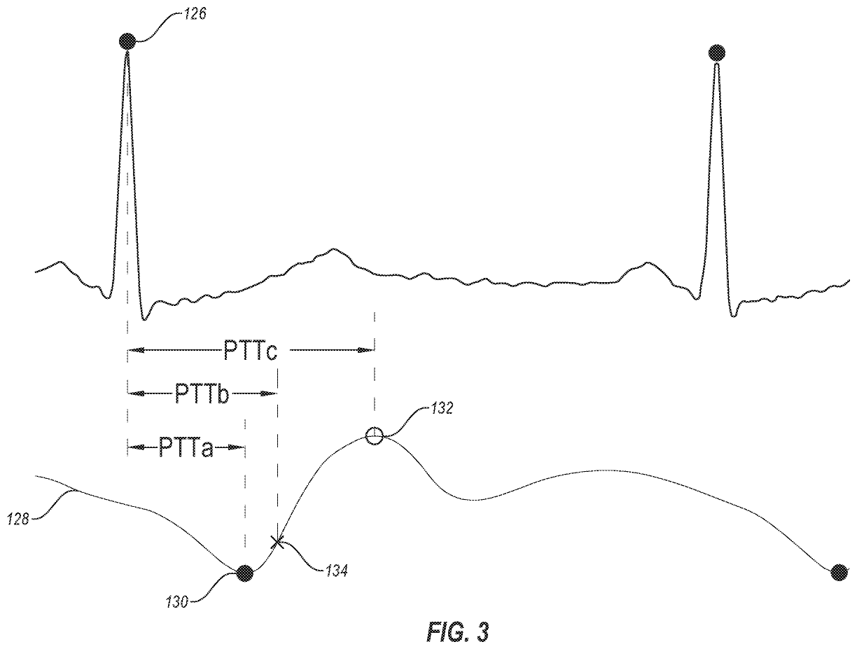

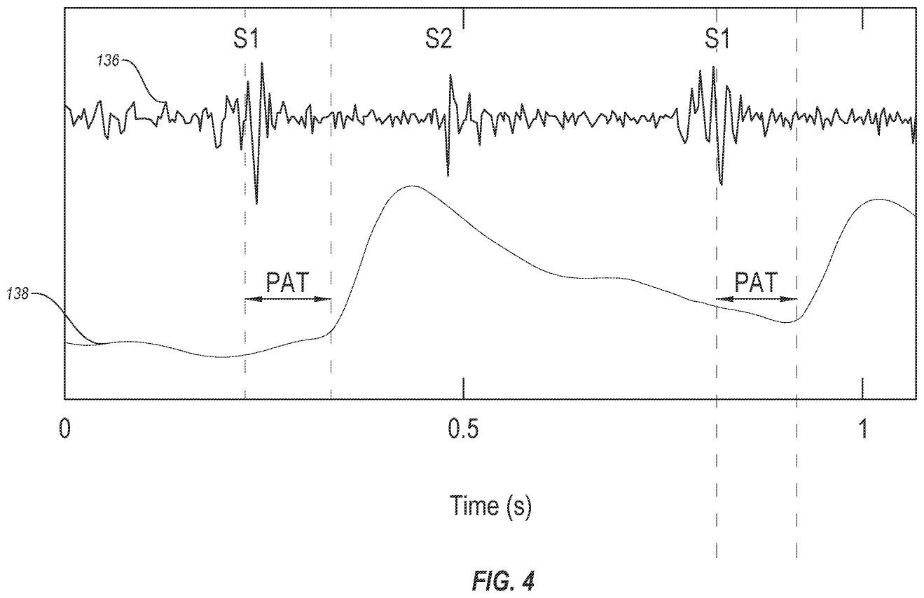

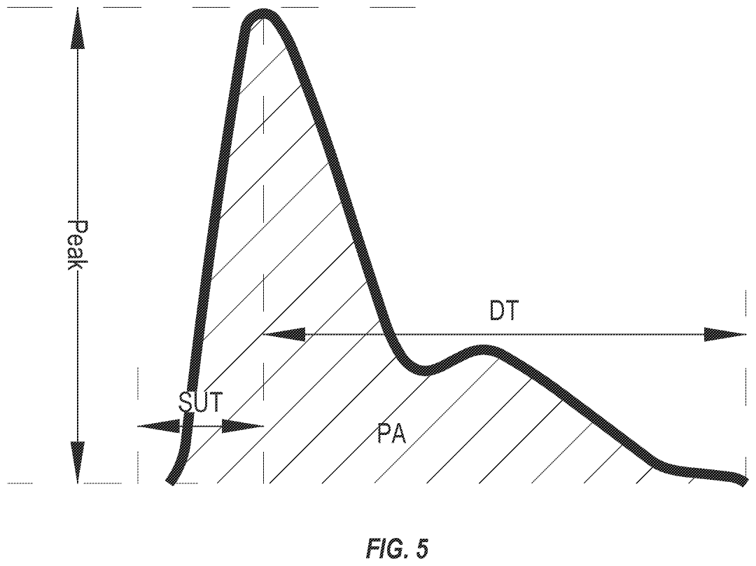

Referring now to FIG. 3, PTT or PAT may for example be estimated from ECG signals obtained with the first sensor 114 by correlating the time delay between an ECG R peak 126 to PPG main wave 128 feature points like the foot 130, peak 132, and a certain portion magnitude of rising edge 134. Moreover, with reference to FIG. 4 for example, PAT may be estimated by correlating signals 136 obtained by the third sensor 118 representative of a first heart sound to features of a PPG main wave 138 like a wave foot. For example, the PAT may be defined as the time delay between a first heart sound (detected for example by the third sensor 118) and a wave foot on the PPG main wave 138. In addition, and with reference to FIG. 5, the system 100 may be further configured to select PPG morphological features that are highly correlated with blood pressure. For example, the Pearson correlation coefficient method may be used to select the PPG morphological features that are highly correlated with systolic blood pressure (SBP) and diastolic blood pressure (DBP). The PPG morphological features which may be extracted from PPG signal segments include, but are not limited to, the pressure constant k (PK) which is related to the total peripheral resistance (TPR) of the blood vessel; the photoplethysmography area (PA) which reflects the TPR and changes in blood vessel tension; the rise time (RT) which reflects the heart contraction and left ventricular function; the descent time (DT) which is related to ventricular diastole; the pulsatile hetero height (PHH) which reflects the magnitude of cardiac output; the pulse wave amplitude (peak); ventricular systolic interval (Ts); and diastolic interval (Td).

In one form, one or more of the sensors 114, 116 and 118 may be configured to acquire pulse-related data from the human patient 112 at a location other than a fingertip, wrist or arm of the human patient 112, although variations are possible. In one form, one or more of the sensors 114, 116 and 118 may be configured to acquire pulse-related data from the wrist of the human patient 112 or from the chest of the human patient 112. In one form, one or more of the sensors 114, 116 and 118 may be provided in a housing having a configuration similar to a wristwatch or a chest strap. In another form, one or more of the sensors 114, 116 and 118 may be provided in a housing having a configuration similar to a hearing aid, or one or more may be included in a hearing aid.

As indicated above, one or more of the sensors 114, 116 and 118 may be configured to transmit data observed and collected thereby to the first processor 120 and/or the second processor 122. In one form, the first processor 120 and the second processor 122 may both be remotely positioned from the human patient 112 and one or more of the sensors 114, 116 and 118. For example, one or both of the first processor 120 and the second processor 122 may be cloud-hosted, although forms in which the first processor 120 is included with or incorporated into the second sensor 116 and/or positioned in the housing 124 are also possible and contemplated. In this latter form for example, the first processor 120 may be configured to transmit blood pressure determinations or estimates made thereby to the second processor 122 and/or a remote data collection network or unit where the blood pressure readings of the human patient 112 may be collected, stored, and accessed for analysis by a caretaker of the human patient 112. In the former form, it is contemplated that blood pressure determinations made by the first processor 120 may be collected, stored, and accessed for analysis by a caretaker of the human patient from a cloud if the first processor 120 is hosted on the cloud, or another suitable data collection medium.

In one form, the first processor 120 may be configured as an edge computing engine structured to apply a light-weighted machine-learning model, a linear regression model or statistical model to the information and data provided by the sensors 114, 116 and 118 in order to estimate the SBP and DBP. In one aspect, the estimated SBP and DBP, extracted features from the information and data provided by the sensors 114, 116 and 118, and the raw data of the information and data provided by the sensors 114, 116 and 118 may be selectively sent to the second processor 122 which may be hosted in the cloud, amongst other possibilities, for a more complicated and powerful deep learning network in order to confirm or otherwise establish values for the SBP and DBP. The second processor 122 may also be configured to apply a light-weighted machine-learning model, a linear regression model or statistical model to the information and it receives in order to determine the SBP and DBP. The blood pressure values determined by the second processor 122 may be provided to a remote data collection network or unit where these blood pressure readings of the human patient 112 may be collected, stored, and accessed for analysis by a caretaker of the human patient 112. In the former form, it is contemplated that blood pressure determinations made by the second processor 122 may be collected, stored, and accessed for analysis by a caretaker of the human patient 112 from the cloud on which the second processor 122 is hosted, or another suitable data collection medium. During operation, it is possible that one or more of the sensors 114, 116 and 118 may observe and record pulse-related data values from the human patient 112 which have less than optimal quality. For example, the information recorded by one or more of the sensors 114, 116 and 118 may be noisy, or poor in quality, which if unaccounted for, may lead to inaccurate blood pressure determinations by the first processor 120 and/or the second processor 122. For example, signal quality associated with one or more of the sensors 114, 116 and 118 may be dependent on the area of the human patient 112 from where one or more of the sensors 114, 116 and 118 obtains data. Use of a sensor in connection with the wrist or chest of the human patient 112 may, for example, provide lower quality signals than would be obtained through use of a sensor in connection with the fingertip of the human patient 112.

In one form, one or more of the sensors 114, 116 and 118 may be configured to discard data or information it obtains from the human patient 112 if one or more of the sensors 114, 116 and 118 determines that the data or information does not meet a predetermined quality threshold. In addition to eliminating consideration of this data or information by the first processor 120 and/or the second processor 122, one or more of the sensors 114, 116 and 118 may also conserve power by foregoing this data transmission. Further, while not previously discussed, one or more of the sensors 114, 116 and 118 may be configured to provide the data and information it records to the first processor 120 and/or the second processor 122 in a raw form, in a compressed form, or a combination of these forms. Additionally or alternatively, one or more of the first processor 120 and the second processor 122 may be configured to discard data or information it receives from one or more of the sensors 114, 116 and 118 if it determines that the data or information does not meet a predetermined quality threshold.

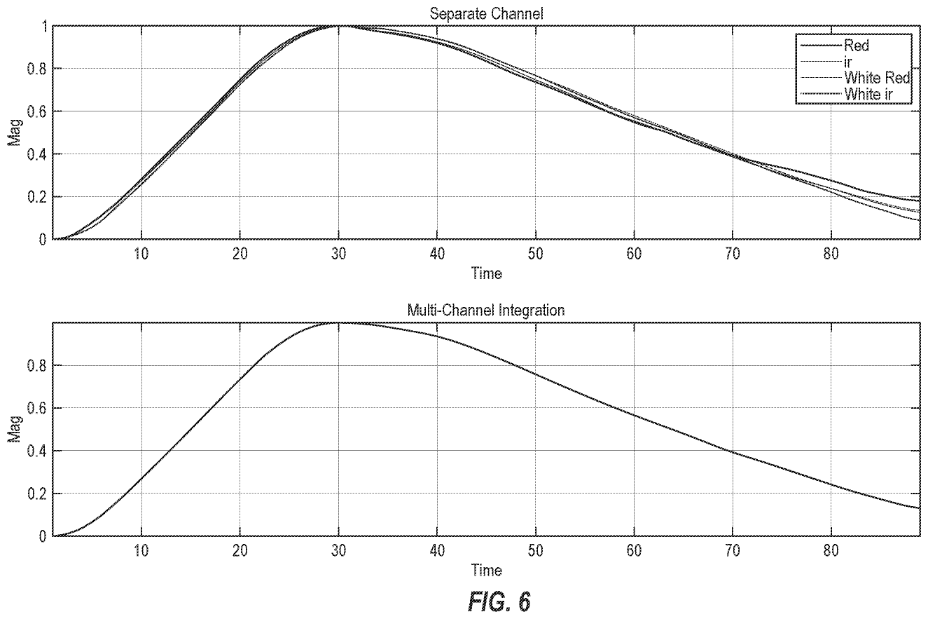

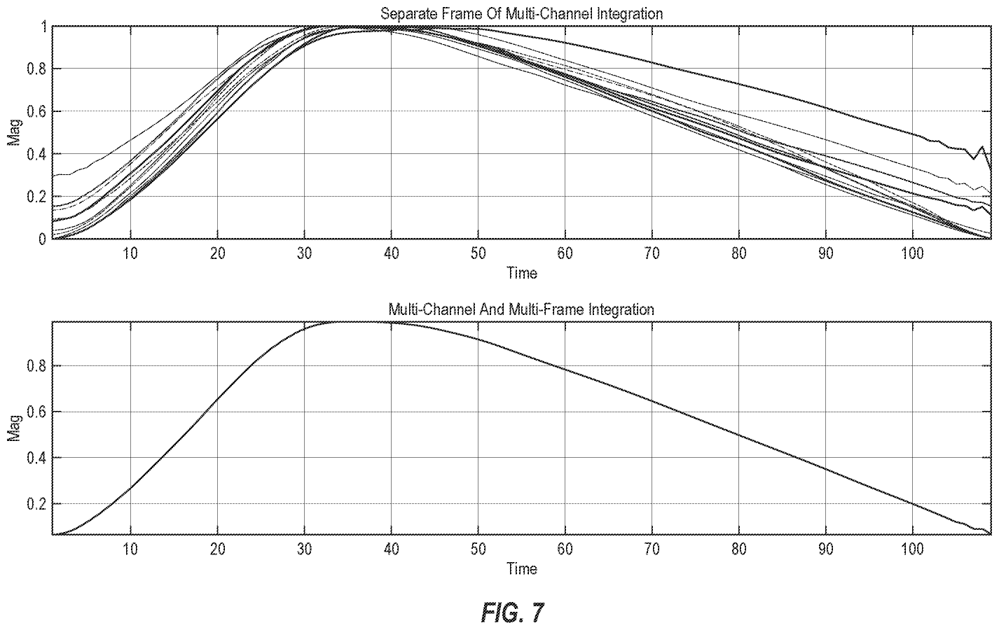

In addition, to account for the possibility of less-than-optimal quality of data provided by one or more of the sensors 114, 116 and 118, the first processor 120 and/or the second processor 122 may be configured to determine, from the various data and information provided by one or more of the sensors 114, 116 and 118, a representative pulse-related data value representative of the pulse-related data values determined by one or more of the sensors 114, 116 and 118 during the period of time of inactivity of the human patient 112. For example, the first processor 120 and/or the second processor 122 may be configured to analyze numerous readings (possibly even on the scale of hundreds or thousands), and attendant data, provided by one or more of the sensors 114, 116 and 118 as observed during the determined period of time of inactivity of the human patient 112, and piece together the data in order to determine data (or a single piece of data or value) that is representative of all of the data/values provided by one or more of the sensors 114, 116 and 118. For example, and with reference to FIGS. 6 and 7, the first processor 120 and/or the second processor 122 may be configured to integrate multi-channel and/or multi-beat data of the PPG sensor over a certain period of time to provide a more robust feature extraction regarding morphologic parameters of the PPG data. The first processor 120 and/or the second processor 122 may then determine a blood pressure value for the human patient during the period of time of inactivity based on the data that is representative of the aggregate data provided by one or more of the sensors 114, 116 and 118. In this regard, the first processor 120 and/or the second processor 122 may account, in the aggregate, for noise or less than optimal quality of the data provided by one or more of the sensors 114, 116 and 118 such that the blood pressure value determined by the first processor 120 and/or the second processor 122 is not somehow based on a small subset of data which may include the same, less than optimal variations. The first processor 120 and/or the second processor 122 may be configured based on a personalized model for the human patient 112, or it may be configured based on a generalized model for all patients.

The system 100 may also be configured to determine and identify changes in blood pressure which may unexpectedly occur during a period of inactivity. For example, in the case of sepsis or other major infection, the human patient 112 could be stationary and remain in the same position while also having significant changes to their blood pressure not due to movement or repositioning. In these instances, when the system 100 detects a change in blood pressure which is unexpected given the period of inactivity, the system 100 may be configured to shorten the interval at which blood pressure determinations are made in order to more closely monitor blood pressure trends over a shorter period or periods of time. For example, the system 100 could determine blood pressure values for thirty- or forty-five-minute periods, just to provide a few examples. If for example the system 100 determines that blood pressure measurements are continually increasing during these intervals, the system 100 may associate these blood pressure changes with physiological deterioration of the human patient 112 and may be configured to transmit a message or alert indicating the same so that medical attention may be provided to the human patient 112. Conversely, if the blood pressure values determined during the period of time exhibit trends returning to, or toward, normal blood pressure values for the human patient 112, then the system 100 may be configured to revert back to normal or previously use periods during which blood pressure is determined.

While not previously discussed, it should be appreciated that the system 100 may be initially calibrated in order to provide future blood pressure values for the human patient 112. For example, the blood pressure of the human patient 112 may be taken using conventional means (such as with a blood pressure cuff) during a period of time of inactivity of the human patient 112, and the measured blood pressure at this time may be correlated and time stamped with pulse-related data recorded by one or more of the sensors 114, 116 and 118 (or a similar sensor) so that future pulse-related data observed and obtained by one or more of the sensors 114, 116 and 118 may be used to determine blood pressure of the human patient 112. In addition, after a period of time (e.g., after several days, a week, or more) has passed after the initial calibration, it is contemplated that the system 100 could be recalibrated in a manner similar to that described in connection with the initial calibration.

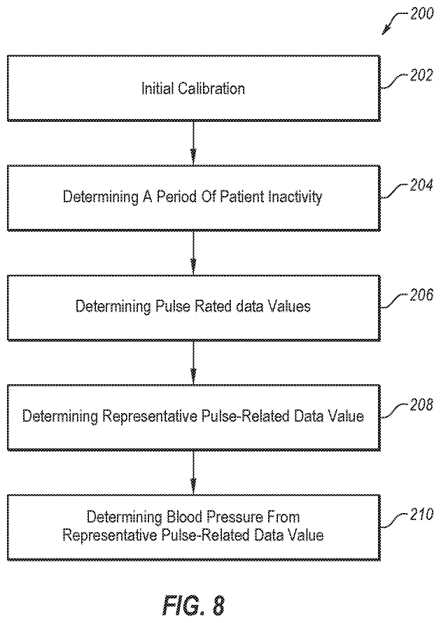

FIG. 8 is a flowchart of an example method 200 for determining blood pressure of a human patient. The method 200 may be used to monitor blood pressure of the human patient remotely (e.g., without physical presence or intervention of a health care professional) over the course of a number of days or weeks. The method 200 may be arranged in accordance with at least one embodiment described in the present disclosure. One or more of the operations of the method 200 may be performed, in some embodiments, by a device or system, such as one or more elements of the system 100 of FIG. 2. In these and other embodiments, at least some portion(s) of the method 200 may be performed based on the execution of instructions stored on one or more non-transitory computer-readable media. Although illustrated as discrete blocks, various blocks may be divided into additional blocks, combined into fewer blocks, or eliminated, depending on the desired implementation.

The method 200 may include block 202 at which an initial calibration may be performed in order to facilitate future determination of blood pressure of a human patient based on observed pulse-related data values. In one form, the block 202 may include taking the blood pressure of the human patient using conventional means (such as with a blood pressure cuff) during a period of time of inactivity of the human patient, and the measured blood pressure at this time may be correlated and time stamped with pulse-related data recorded by one or more sensors, such as one or more of the sensors 114, 116 and 118 (or a similar sensor), so that future pulse-related data observed and obtained by a sensor, such as one or more of the sensors 114, 116 and 118, may be used to determine blood pressure of the human patient.

At block 204, a period of time during which the human patient is stationary or inactive may be determined. This determination may be made with one or more sensors associated with the human patient in a manner described herein above. For example, one or more motion sensors such as accelerometers may be configured to detect movement, or a lack of movement, of the patient. The sensor may be further configured to determine the length of the period of time during which the patient is in active.

At the block 206, a plurality of measurements providing pulse-related data values from the human patient may be determined. In one aspect, these measurements may occur or be obtained during the period of time of inactivity determined at the block 204, or during at least portion thereof. In one form, the measurements may be made utilizing a relevant sensor such as one or more of the sensors 114, 116 and 118. In one form, the pulse-related data values may include one or more pulse waveforms and/or or one or more portions of one or more pulse waveforms.

At the block 208, a representative pulse-related data value representative of the pulse-related data values determined during the determined period of time of inactivity may be determined from the plurality of measurements providing pulse-related data values. For example, the representative pulse-related data value may be determined by a processor such as the first processor 120 and/or the second processor 122 described herein above. At the block 210, a blood pressure value for the human being during the period of time of inactivity may be determined from the representative pulse-related data value.

Modifications, additions, or omissions may be made to the method 200 without departing from the scope of the present disclosure. For example, some of the operations of method 200 may be implemented in differing order. Additionally or alternatively, two or more operations may be performed at the same time. Furthermore, the outlined operations and actions are only provided as examples, and some of the operations and actions may be optional, combined into fewer operations and actions, or expanded into additional operations and actions without detracting from the essence of the disclosed embodiments. For example, in some embodiments, the method 200 may include one or more of the operations described above with respect to FIG. 2.

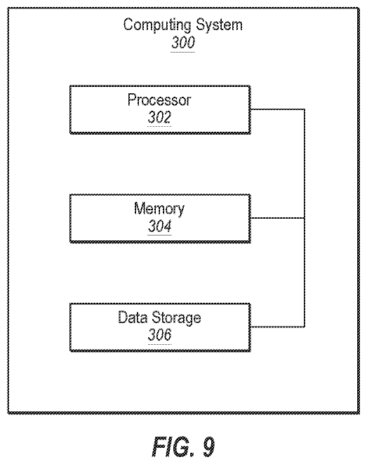

FIG. 9 illustrates a block diagram of an example computing system 300. The computing system 300 may be configured according to at least one embodiment of the present disclosure and may be an example of computing systems that may include or be part of one or more elements of the system 100 of FIG. 2. For example, the system 100 may include one or more computing systems 300. For instance, the computing system 300 may be an example of the first processor 120 and/or the second processor 122 of FIG. 2. The computing system 300 may include a processor 302, a memory 304, and a data storage 306. The processor 302, the memory 304, and the data storage 306 may be communicatively coupled. The processor 302 may correspond to the first processor 120 and/or the second processor 122 described herein, and the data storage 306 may correspond to the database or data collection unit described herein.

In general, the processor 302 may include any suitable special-purpose or general-purpose computer, computing entity, or processing device including various computer hardware or software modules and may be configured to execute instructions stored on any applicable computer-readable storage media. For example, the processor 302 may include a microprocessor, a microcontroller, a digital signal processor (DSP), an application-specific integrated circuit (ASIC), a Field-Programmable Gate Array (FPGA), or any other digital or analog circuitry configured to interpret and/or to execute program instructions and/or to process data. Although illustrated as a single processor in FIG. 9, the processor 302 may include any number of processors configured to, individually or collectively, perform or direct performance of any number of operations described in the present disclosure. Additionally, one or more of the processors may be present on one or more different electronic devices, such as different servers.

In some embodiments, the processor 302 may be configured to interpret and/or execute program instructions and/or process data stored in the memory 304, the data storage 306, or the memory 304 and the data storage 306. In some embodiments, the processor 302 may fetch program instructions from the data storage 306 and load the program instructions in the memory 304. After the program instructions are loaded into memory 304, the processor 302 may execute the program instructions.

The memory 304 and the data storage 306 may include computer-readable storage media for carrying or having computer-executable instructions or data structures stored thereon. Such computer-readable storage media may include any available media that may be accessed by a general-purpose or special-purpose computer, such as the processor 302. By way of example, and not limitation, such computer-readable storage media may include tangible or non-transitory computer-readable storage media including Random Access Memory (RAM), Read-Only Memory (ROM), Electrically Erasable Programmable Read-Only Memory (EEPROM), Compact Disc Read-Only Memory (CD-ROM) or other optical disk storage, magnetic disk storage or other magnetic storage devices, flash memory devices (e.g., solid state memory devices), or any other storage medium which may be used to store program code in the form of computer-executable instructions or data structures and which may be accessed by a general-purpose or special-purpose computer. In these and other embodiments, the term “non-transitory” as explained herein should be construed to exclude only those types of transitory media that were found to fall outside the scope of patentable subject matter in the Federal Circuit decision of In re Nuijten, 500 F.3d 1346 (Fed. Cir. 2007). Combinations of the above may also be included within the scope of computer-readable media.

Modifications, additions, or omissions may be made to the computing system 300 without departing from the scope of the present disclosure. For example, in some embodiments, the computing system 300 may include any number of other components that may not be explicitly illustrated or described.

For instance, in some embodiments, the computing system 300 may include a communication unit that includes any component, device, system, or combination thereof that is configured to transmit or receive information over a network. In some embodiments, the communication unit may communicate with other devices at other locations, the same location, or even other components within the same system. For example, the communication unit may include a modem, a network card (wireless or wired), an optical communication device, an infrared communication device, a wireless communication device (such as an antenna), and/or chipset (such as a Bluetooth device, an 802.6 device (e.g., Metropolitan Area Network (MAN)), a Wi-Fi device, a WiMax device, cellular communication facilities, or others), and/or the like. The communication unit may permit data to be exchanged with a network and/or any other devices or systems described in the present disclosure. For example, the communication unit may allow the computing system 300 to communicate with other systems, such as computing devices and/or other networks.

Additionally or alternatively, the computing system 300 may include one or more user interfaces in some embodiments. The user interfaces may include any system or device to allow a user to interface with the computing system 300. For example, the interfaces may include a mouse, a track pad, a keyboard, and/or a touchscreen, among other devices or systems. The interfaces may also include a graphical user interface that may be presented on a display that may be included with the computing system 300. The display may be configured as one or more displays, like an LCD, LED, or other type of display. The display may be configured to present content such as video, text, user interfaces, and other data as directed by the processor.

As indicated above, the embodiments described in the present disclosure may include the use of a special purpose or general-purpose computer (e.g., the processor 302 of FIG. 9) including various computer hardware or software modules, as discussed in greater detail below. Further, as indicated above, embodiments described in the present disclosure may be implemented using computer-readable media (e.g., the memory 304 or data storage 306 of FIG. 9) for carrying or having computer-executable instructions or data structures stored thereon.

In some embodiments, the components of the system 100 may communicate over a network. The network may include a short-range wireless network, such as a wireless local area network (WLAN), a personal area network (PAN), or a wireless mesh network (WMN). For example, the network may include networks that use Bluetooth® Class 2 and Class 3 communications with protocols that are managed by the Bluetooth® Special Interest Group (SIG). Other examples of wireless networks may include the IEEE 802.11 networks (commonly referred to as Wi-Fi®), Zigbee networks, among other types of LANS, PANS, and WMNS. In these or other embodiments, the network may include a wide area network (WAN) that may extend over a relatively large geographic area as compared to the geographic area that may be covered by a short-range wireless network. In some embodiments, the network may have numerous different configurations. In some embodiments, the network may include a peer-to-peer network.

Additionally or alternatively, the network may also be coupled to or may include portions of a telecommunications network for sending data in a variety of different communication protocols. In some embodiments, the network may include an Internet Protocol (IP) based network such as the Internet. In some embodiments, the network may include cellular communication networks for sending and receiving communications and/or data including via hypertext transfer protocol (HTTP), direct data connection, wireless application protocol (WAP), etc. The network may also include a mobile data network that may include third-generation (3G), fourth-generation (4G), fifth-generation (5G), long-term evolution (LTE), long-term evolution advanced (LTE-A), Voice-over-LTE, Voice-over-IP, or any other mobile data network or combination of mobile data networks.

In one embodiment, a method for determining a blood pressure value of a human being includes determining a period of time during which the human being is stationary; determining, during the period of time, a plurality of measurements providing pulse-related data values from the human being; determining, from the plurality of measurements, a representative pulse-related data value representative of the pulse-related data values determined during the period of time; and determining, from the representative pulse-related data value, a blood pressure value for the human being during the period of time. In one form, the method further includes determining if one or more of the plurality of measurements providing pulse-related data values is below a threshold quality and discarding any of the one or more of the measurements providing pulse-related data determined to be below the threshold quality.

In another form, the plurality of measurements providing pulse-related data values of the human being are taken from a location on the human being other than a fingertip, wrist, or arm of the human being.

In still another form, the plurality of measurements providing pulse-related data values of the human being are taken from a wrist or chest of the human being.

In yet another form, determining the plurality of measurements providing pulse-related data values of the human being includes measuring pulse-related values of the human being with one or more of at least one electrocardiogram (ECG) sensor, at least one photoplethysmogram (PPG) sensor, at least one accelerometer, and at least one acoustic sensor.

In one aspect of this form, the method further includes transmitting at least a portion of the plurality of measurements providing pulse-related data values of the human being from one or more of the at least one electrocardiogram (ECG) sensor, the at least one photoplethysmogram (PPG) sensor, the at least one accelerometer, and the at least one acoustic sensor to a server.

In a further aspect, the transmitting includes providing one or both of a compressed version of the portion of the plurality of measurements providing pulse-related data values of the human being and a raw version of the portion of the plurality of measurements providing pulse-related data values of the human being.

In yet another aspect, at least one of the server and one or more of the at least one electrocardiogram (ECG) sensor, the at least one photoplethysmogram (PPG) sensor, the at least one accelerometer, and the at least one acoustic sensor is configured to discard one or more of the plurality of measurements providing pulse-related data values which fall below a threshold quality.

In another aspect, determining the plurality of measurements providing pulse-related data values of the human being includes determining ventricular contraction of the heart based on measurements taken by one or more of the at least one electrocardiogram (ECG) sensor, the at least one photoplethysmogram (PPG) sensor, the at least one accelerometer, and the at least one acoustic sensor.

In another form, determining the period of time during which the human being is stationary includes at least one of measuring motion of the human being and determining positioning of the human being.

In still another form, the method further includes selecting a number of the plurality of measurements providing pulse-related data values satisfying a threshold quality over a segment of time and averaging the number of the plurality of measurements providing pulse-related data values for the segment of time.

In another form, the plurality of measurements providing pulse-related data values from the human being include a number of multi-channel and multi-beat measurements from a photoplethysmogram (PPG) sensor over a certain period of time, and the number of multi-channel and multi-beat measurements from the PPG sensor over the certain period of time are integrated.

In still another form, the method further includes applying a machine-learning model or a linear regression model with an edge computing engine for at least one of determining the representative pulse-related data value and determining the blood pressure value for the human being during the period of time.

In one aspect, the method further includes applying a cloud-based machine-learning model or a linear regression model for at least one of determining the representative pulse-related data value and determining the blood pressure value for the human being during the period of time.

In one form, the method further includes applying at least one of a cloud-based machine-learning model, a linear regression model and a statistical model for at least one of determining the representative pulse-related data value and determining the blood pressure value for the human being during the period of time.

In another form, the method further includes applying a machine-learning model or a linear regression model with an edge computing engine for determining an estimated blood pressure value for the human being during the period of time.

In one aspect of this form, the method further includes providing the estimated blood pressure value for the human being during the period of time to a cloud-based determination module and determining the blood pressure value for the human being during the period of time with the cloud-based determination module.

In another embodiment, a system for determining a blood pressure value of a human being includes a first sensor configured to determine a period of time during which the human being is stationary; at least one pulse-related sensor configured to determine a plurality of measurements providing pulse-related data values of the human being during the period of time; and a processor configured to determine, from the plurality of measurements, a representative pulse-related data value representative of the plurality of pulse-related data values determined during the period of time, and to determine, from the representative pulse-related data value, a blood pressure value for the human being during the period of time.

In one form, the first sensor is an accelerometer, and the first sensor and the at least one pulse-related sensor are positioned in a common housing.

In another form, the at least one pulse-related sensor is configured to determine the plurality of measurements providing pulse-related data values of the human being during the period of time in response to a determination by the first sensor that the human being is stationary.

In still another form, each of the plurality of measurements providing pulse-related data values includes at least a portion of a pulse waveform.

In yet another form, the at least one pulse-related sensor includes one or more of an electrocardiogram (ECG) sensor, a photoplethysmogram (PPG) sensor, an acoustic sensor, and an accelerometer.

In still another form, the at least one pulse-related sensor is configured to determine ventricular contraction of the heart based on measurements taken by an accelerometer and an acoustic sensor.

In a further form, the processor is configured to select a number of the plurality of measurements providing pulse-related data values satisfying a threshold quality over a segment of time and average the number of the plurality of measurements providing pulse-related data values for the segment of time.

In still another form, the plurality of measurements providing pulse-related data values from the human being include a number of multi-channel and multi-beat measurements from a photoplethysmogram (PPG) sensor over a certain period of time, and the processor is configured to integrate the number of multi-channel and multi-beat measurements from the PPG sensor over the certain period.

In yet another form, the processor is configured to apply a machine-learning model or a linear regression model to determine, from the plurality of measurements, the representative pulse-related data value representative of the plurality of pulse-related data values determined during the period of time, and to determine, from the representative pulse-related data value, the blood pressure value for the human being during the period of time.

In some embodiments, the different components, modules, engines, and services described herein may be implemented as objects or processes that execute on a computing system (e.g., as separate threads). While some of the systems and methods described in the present disclosure are generally described as being implemented in software (stored on and/or executed by general purpose hardware), specific hardware implementations or a combination of software and specific hardware implementations are also possible and contemplated.

In accordance with common practice, the various features illustrated in the drawings may not be drawn to scale. The illustrations presented in the present disclosure are not meant to be actual views of any particular apparatus (e.g., device, system, etc.) or method, but are merely idealized representations that are employed to describe various embodiments of the disclosure. Accordingly, the dimensions of the various features may be arbitrarily expanded or reduced for clarity. In addition, some of the drawings may be simplified for clarity. Thus, the drawings may not depict all of the components of a given apparatus (e.g., device) or all operations of a particular method.

Terms used in the present disclosure and especially in the appended claims (e.g., bodies of the appended claims) are generally intended as “open” terms (e.g., the term “including” should be interpreted as “including, but not limited to,” the term “having” should be interpreted as “having at least,” the term “includes” should be interpreted as “includes, but is not limited to,” etc.).

Additionally, if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations.

In addition, even if a specific number of an introduced claim recitation is explicitly recited, such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” or “one or more of A, B, and C, etc.” is used, in general such a construction is intended to include A alone, B alone, C alone, A and B together, A and C together, B and C together, or A, B, and C together, etc. For example, the use of the term “and/or” is intended to be construed in this manner.

Further, any disjunctive word or phrase presenting two or more alternative terms, whether in the description, claims, or drawings, should be understood to contemplate the possibilities of including one of the terms, either of the terms, or both terms. For example, the phrase “A or B” should be understood to include the possibilities of “A” or “B” or “A and B.”

Additionally, the use of the terms “first,” “second,” “third,” etc., are not necessarily used in the present disclosure to connote a specific order or number of elements. Generally, the terms “first,” “second,” “third,” etc., are used to distinguish between different elements as generic identifiers. Absence a showing that the terms “first,” “second,” “third,” etc., connote a specific order, these terms should not be understood to connote a specific order. Furthermore, absence a showing that the terms first,” “second,” “third,” etc., connote a specific number of elements, these terms should not be understood to connote a specific number of elements. For example, a first widget may be described as having a first side and a second widget may be described as having a second side. The use of the term “second side” with respect to the second widget may be to distinguish such side of the second widget from the “first side” of the first widget and not to connote that the second widget has two sides.

All examples and conditional language recited in the present disclosure are intended for pedagogical objects to aid the reader in understanding the invention and the concepts contributed by the inventor to furthering the art, and are to be construed as being without limitation to such specifically recited examples and conditions. Although embodiments of the present disclosure have been described in detail, it should be understood that the various changes, substitutions, and alterations could be made hereto without departing from the spirit and scope of the present disclosure.

What is claimed is:

1. A method for determining a blood pressure value of a human being, comprising:
determining a period of time during which the human being is stationary;
determining, during the period of time, a plurality of measurements providing pulse-related data values from the human being;
determining, from the plurality of measurements, a representative pulse-related data value representative of the pulse-related data values determined during the period of time; and
determining, from the representative pulse-related data value, a blood pressure value for the human being during the period of time.

2. The method of claim 1, further comprising determining if one or more of the plurality of measurements providing pulse-related data values is below a threshold quality and discarding any of the one or more of the measurements providing pulse-related data determined to be below the threshold quality.

3. The method of claim 1, wherein the plurality of measurements providing pulse-related data values of the human being are taken from a location on the human being other than a fingertip, wrist, or arm of the human being.

4. The method of claim 1, wherein the plurality of measurements providing pulse-related data values of the human being are taken from a wrist or chest of the human being.

5. The method of claim 1, wherein determining the plurality of measurements providing pulse-related data values of the human being includes measuring pulse-related values of the human being with one or more of at least one electrocardiogram (ECG) sensor, at least one photoplethysmogram (PPG) sensor, at least one accelerometer, and at least one acoustic sensor.

6. The method of claim 5, further comprising transmitting at least a portion of the plurality of measurements providing pulse-related data values of the human being from one or more of the at least one electrocardiogram (ECG) sensor, the at least one photoplethysmogram (PPG) sensor, the at least one accelerometer, and the at least one acoustic sensor to a server.

7. The method of claim 6, wherein the transmitting includes providing one or both of a compressed version of the portion of the plurality of measurements providing pulse-related data values of the human being and a raw version of the portion of the plurality of measurements providing pulse-related data values of the human being.

8. The method of claim 7, wherein at least one of the server and one or more of the at least one electrocardiogram (ECG) sensor, the at least one photoplethysmogram (PPG) sensor, the at least one accelerometer, and the at least one acoustic sensor is configured to discard one or more of the plurality of measurements providing pulse-related data values which fall below a threshold quality.

9. The method of claim 5, wherein determining the plurality of measurements providing pulse-related data values of the human being includes determining ventricular contraction of the heart based on measurements taken by one or more of the at least one electrocardiogram (ECG) sensor, the at least one photoplethysmogram (PPG) sensor, the at least one accelerometer, and the at least one acoustic sensor.

10. The method of claim 1, wherein determining the period of time during which the human being is stationary includes at least one of measuring motion of the human being and determining positioning of the human being.

11. The method of claim 1, further comprising selecting a number of the plurality of measurements providing pulse-related data values satisfying a threshold quality over a segment of time and averaging the number of the plurality of measurements providing pulse-related data values for the segment of time.

12. The method of claim 1, wherein the plurality of measurements providing pulse-related data values from the human being include a number of multi-channel and multi-beat measurements from a photoplethysmogram (PPG) sensor over a certain period of time, and the number of multi-channel and multi-beat measurements from the PPG sensor over the certain period of time are integrated.

13. The method of claim 1, further comprising applying a machine-learning model or a linear regression model with an edge computing engine for at least one of determining the representative pulse-related data value and determining the blood pressure value for the human being during the period of time.

14. The method of claim 13, further comprising applying a cloud-based machine-learning model or a linear regression model for at least one of determining the representative pulse-related data value and determining the blood pressure value for the human being during the period of time.

15. The method of claim 1, further comprising applying at least one of a cloud-based machine-learning model, a linear regression model and a statistical model for at least one of determining the representative pulse-related data value and determining the blood pressure value for the human being during the period of time.

16. The method of claim 1, further comprising applying a machine-learning model or a linear regression model with an edge computing engine for determining an estimated blood pressure value for the human being during the period of time.

17. The method of claim 16, further comprising providing the estimated blood pressure value for the human being during the period of time to a cloud-based determination module and determining the blood pressure value for the human being during the period of time with the cloud-based determination module.

18. A system for determining a blood pressure value of a human being, comprising:
a first sensor configured to determine a period of time during which the human being is stationary;
at least one pulse-related sensor configured to determine a plurality of measurements providing pulse-related data values of the human being during the period of time; and
a processor configured to determine, from the plurality of measurements, a representative pulse-related data value representative of the plurality of pulse-related data values determined during the period of time, and to determine, from the representative pulse-related data value, a blood pressure value for the human being during the period of time.

19. The system of claim 18, wherein the first sensor is an accelerometer, and the first sensor and the at least one pulse-related sensor are positioned in a common housing.

20. The system of claim 18, wherein the at least one pulse-related sensor is configured to determine the plurality of measurements providing pulse-related data values of the human being during the period of time in response to a determination by the first sensor that the human being is stationary.

21. The system of claim 18, wherein each of the plurality of measurements providing pulse-related data values includes at least a portion of a pulse waveform.

22. The system of claim 18, wherein the at least one pulse-related sensor includes one or more of an electrocardiogram (ECG) sensor, a photoplethysmogram (PPG) sensor, an acoustic sensor, and an accelerometer.

23. The system of claim 18, wherein the at least one pulse-related sensor is configured to determine ventricular contraction of the heart based on measurements taken by an accelerometer and an acoustic sensor.

24. The system of claim 18, wherein the processor is configured to select a number of the plurality of measurements providing pulse-related data values satisfying a threshold quality over a segment of time and average the number of the plurality of measurements providing pulse-related data values for the segment of time.

25. The system of claim 18, wherein the plurality of measurements providing pulse-related data values from the human being include a number of multi-channel and multi-beat measurements from a photoplethysmogram (PPG) sensor over a certain period of time, and the processor is configured to integrate the number of multi-channel and multi-beat measurements from the PPG sensor over the certain period.

26. The system of claim 18, wherein the processor is configured to apply a machine-learning model or a linear regression model to determine, from the plurality of measurements, the representative pulse-related data value representative of the plurality of pulse-related data values determined during the period of time, and to determine, from the representative pulse-related data value, the blood pressure value for the human being during the period of time.
